(19) United States
(12) Patent Application Publication
(10) Pub. No.: US 2025/0259713 A1
(43) Pub. Date: Aug. 14, 2025

(54) Library Search Using Deep Learning Based Spectral Compression

(71) Applicant: DH Technologies Development Pte. Ltd., Singapore (SG)

(72) Inventor: Fras WASIM, Mississauga (CA)

(21) Appl. No.: 18/856,371

(22) PCT Filed: Apr. 4, 2023
(86) PCT No.: PCT/IB2023/053429

§ 371 Date: Oct. 11, 2024

Related U.S. Application Data
(60) Provisional application No. 63/362,833, filed on Apr. 12, 2022.

(51) Int. Cl. G16C 20/20; G06N 3/044; G06N 3/088; G06N 3/09; G16C 20/64; G16C 20/70

(52) U.S. Cl. CPC G16C 20/20; G06N 3/044; G06N 3/088; G06N 3/09; G16C 20/64; G16C 20/70

(57) ABSTRACT

Known mass spectral data of a library of spectra corresponding to known compounds or known mass spectral data determined from a database of known compounds are compressed using a neural network encoder, producing a group of corresponding compressed known representations of known mass spectral data. Experimental mass spectral data of an experimental mass spectrum is compressed using the neural network encoder, producing a compressed experimental representation of the experimental mass spectral data. The experimental representation is compared to the group of known representations and each comparison is scored. At least one comparison with a score above a predetermined score threshold is selected. A known compound is determined from the selected at least one comparison. The known compound is identified as a compound of the experimental spectrum.

### RELATED APPLICATIONS

This application claims the benefit of U.S. Provisional Patent Application Ser. No. 63/362,833, filed on Apr. 12, 2022, the content of which is incorporated by reference herein in its entirety.

### FIELD

The teachings herein relate to identifying a compound of an experimental mass spectrum using a library or database search.

### INTRODUCTION

### Library Search Complexity Limitation

Tandem mass spectrometry (or mass spectrometry/mass spectrometry MS/MS), which is described below, is often used to identify a compound of an experimental sample. Typically, an MS/MS or product ion spectrum is obtained. Then, some spectral data from the product ion spectrum is compared to spectral data of each mass spectrum of a library of spectra corresponding to known compounds. Alternatively, the spectral data from the product ion spectrum is compared to spectral data determined from each compound of a database of known compounds. Spectral data is determined from a database of known compounds by using in silico or theoretical fragmentation of each compound in the database, for example.

The spectral data compared to the library of spectra or database of known compounds includes, for example, mass-to-charge ratio (m/z) values. This spectral data can also include, but is not limited to, intensity values, retention times, and precursor ion data.

Each comparison of spectral data is scored. Many different techniques are used conventionally to score spectral data. For example, cosine scoring provides a score that is a measure of the similarity of the intensity and m/z values. Alternatively, spec2vec, for example, provides a score based on relationships among fragments just like natural language processing algorithms can compare two sentences by also understanding the relationships between words within each sentence.

Once all the comparisons are scored, one or more compounds are selected based on the scores. The one or more selected compounds are then identified as a possible compound of the experimental sample.

One of the reasons that MS/MS is used in compound identification is that the fragmentation provides additional information on the chemical structure of the compound. In other words, fragmentation is used to determine the differences in the chemical structure of compounds.

MS/MS identification using library or database search, therefore, works particularly well when the compounds of a sample vary significantly in chemical structure, particularly when the fragments of different compounds in the sample vary significantly. Unfortunately, when a sample becomes more complex, more and more of the compounds in the sample have similar fragments. As a result, MS/MS identification using library or database search becomes more difficult for more complex samples.

In recent years, there has been increasing work on MS/MS identification using library or database search for small molecules where complexity limits the quality of results achievable. In addition, the size of libraries and databases has also been increasing and current solutions require significant compute power, via the use of cloud computing, or have limited library size if run locally.

As a result, additional systems and methods are needed to improve the results and reduce the memory and computer processing needed to identify small molecules in complex experimental samples using MS/MS and library or database search.

### LC-MS and LC-MS/MS Background

Mass spectrometry (MS) is an analytical technique for the detection and quantitation of chemical compounds based on the analysis of mass-to-charge ratios (m/z) of ions formed from those compounds. The combination of mass spectrometry (MS) and liquid chromatography (LC) is an important analytical tool for the identification and quantitation of compounds within a mixture. Generally, in liquid chromatography, a fluid sample under analysis is passed through a column filled with a chemically-treated solid adsorbent material (typically in the form of small solid particles, e.g., silica). Due to slightly different interactions of components of the mixture with the solid adsorbent material (typically referred to as the stationary phase), the different components can have different transit (elution) times through the packed column, resulting in separation of the various components.

Note that the terms “mass” and “m/z” are used interchangeably herein. One of ordinary skill in the art understands that a mass can be found from an m/z by multiplying the m/z by the charge. Similarly, the m/z can be found from a mass by dividing the mass by the charge.

In LC-MS, the effluent exiting the LC column can be continuously subjected to MS analysis. The data from this analysis can be processed to generate an extracted ion chromatogram (XIC), which can depict detected ion intensity (a measure of the number of detected ions of one or more particular analytes) as a function of retention time.

In MS analysis, an MS or precursor ion scan is performed at each interval of the separation for a mass range that includes the precursor ion. An MS scan includes the selection of a precursor ion or precursor ion range and mass analysis of the precursor ion or precursor ion range.

In some cases, the LC effluent can be subjected to tandem mass spectrometry (or mass spectrometry/mass spectrometry MS/MS) for the identification of product ions corresponding to the peaks in the XIC. For example, the precursor ions can be selected based on their mass/charge ratio to be subjected to subsequent stages of mass analysis. For example, the selected precursor ions can be fragmented (e.g., via collision-induced dissociation), and the fragmented ions (product ions) can be analyzed via a subsequent stage of mass spectrometry.

### Tandem Mass Spectrometry or MS/MS Background

Tandem mass spectrometry or MS/MS involves ionization of one or more compounds of interest from a sample, selection of one or more precursor ions of the one or more compounds, fragmentation of the one or more precursor ions into product ions, and mass analysis of the product ions.

Tandem mass spectrometry can provide both qualitative and quantitative information. The product ion spectrum can be used to identify a molecule of interest. The intensity of one or more product ions can be used to quantitate the amount of the compound present in a sample.

A large number of different types of experimental methods or workflows can be performed using a tandem mass spectrometer. These workflows can include, but are not limited to, targeted acquisition, information dependent acquisition (IDA) or data dependent acquisition (DDA), and data independent acquisition (DIA).

In a targeted acquisition method, one or more transitions of a precursor ion to a product ion are predefined for a compound of interest. As a sample is being introduced into the tandem mass spectrometer, the one or more transitions are interrogated during each time period or cycle of a plurality of time periods or cycles. In other words, the mass spectrometer selects and fragments the precursor ion of each transition and performs a targeted mass analysis for the product ion of the transition. As a result, a chromatogram (the variation of the intensity with retention time) is produced for each transition. Targeted acquisition methods include, but are not limited to, multiple reaction monitoring (MRM) and selected reaction monitoring (SRM).

MRM experiments are typically performed using “low resolution” instruments that include, but are not limited to, triple quadrupole (QqQ) or quadrupole linear ion trap (QqLIT) devices. With the advent of “high resolution” instruments, there was a desire to collect MS and MS/MS using workflows that are similar to QqQ/QqLIT systems. High-resolution instruments include, but are not limited to, quadrupole time-of-flight (QqTOF) or orbitrap devices. These high-resolution instruments also provide new functionality.

MRM on QqQ/QqLIT systems is the standard mass spectrometric technique of choice for targeted quantification in all application areas, due to its ability to provide the highest specificity and sensitivity for the detection of specific components in complex mixtures. However, the speed and sensitivity of today's accurate mass systems have enabled a new quantification strategy with similar performance characteristics. In this strategy (termed MRM high resolution (MRM-HR) or parallel reaction monitoring (PRM)), looped MS/MS spectra are collected at high-resolution with short accumulation times, and then fragment ions (product ions) are extracted post-acquisition to generate MRM-like peaks for integration and quantification. With instrumentation like the TRIPLETOF® Systems of AB SCIEX™, this targeted technique is sensitive and fast enough to enable quantitative performance similar to higher-end triple quadrupole instruments, with full fragmentation data measured at high resolution and high mass accuracy.

In other words, in methods such as MRM-HR, a high-resolution precursor ion mass spectrum is obtained, one or more precursor ions are selected and fragmented, and a high-resolution full product ion spectrum is obtained for each selected precursor ion. A full product ion spectrum is collected for each selected precursor ion but a product ion mass of interest can be specified and everything other than the mass window of the product ion mass of interest can be discarded.

In an IDA (or DDA) method, a user can specify criteria for collecting mass spectra of product ions while a sample is being introduced into the tandem mass spectrometer. For example, in an IDA method a precursor ion or mass spectrometry (MS) survey scan is performed to generate a precursor ion peak list. The user can select criteria to filter the peak list for a subset of the precursor ions on the peak list. The survey scan and peak list are periodically refreshed or updated, and MS/MS is then performed on each precursor ion of the subset of precursor ions. A product ion spectrum is produced for each precursor ion. MS/MS is repeatedly performed on the precursor ions of the subset of precursor ions as the sample is being introduced into the tandem mass spectrometer.

In proteomics and many other applications, however, the complexity and dynamic range of compounds is very large. This poses challenges for traditional targeted and IDA methods, requiring very high-speed MS/MS acquisition to deeply interrogate the sample in order to both identify and quantify a broad range of analytes.

As a result, DIA methods, the third broad category of tandem mass spectrometry, were developed. These DIA methods have been used to increase the reproducibility and comprehensiveness of data collection from complex samples. DIA methods can also be called non-specific fragmentation methods. In a DIA method the actions of the tandem mass spectrometer are not varied among MS/MS scans based on data acquired in a previous precursor or survey scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

The precursor ion mass selection window used to scan the mass range can be narrow so that the likelihood of multiple precursors within the window is small. This type of DIA method is called, for example, MS/MS^{ALL.} In an MS/MS^{ALL} method, a precursor ion mass selection window of about 1 Da is scanned or stepped across an entire mass range. A product ion spectrum is produced for each 1 Da precursor mass window. The time it takes to analyze or scan the entire mass range once is referred to as one scan cycle. Scanning a narrow precursor ion mass selection window across a wide precursor ion mass range during each cycle, however, can take a long time and is not practical for some instruments and experiments.

As a result, a larger precursor ion mass selection window, or selection window with a greater width, is stepped across the entire precursor mass range. This type of DIA method is called, for example, SWATH acquisition. In a SWATH acquisition, the precursor ion mass selection window stepped across the precursor mass range in each cycle may have a width of 5-25 Da, or even larger. Like the MS/MS^{ALL} method, all of the precursor ions in each precursor ion mass selection window are fragmented, and all of the product ions of all of the precursor ions in each mass selection window are mass analyzed. However, because a wider precursor ion mass selection window is used, the cycle time can be significantly reduced in comparison to the cycle time of the MS/MS^{ALL} method.

U.S. Pat. No. 8,809,770 describes how SWATH acquisition can be used to provide quantitative and qualitative information about the precursor ions of compounds of interest. In particular, the product ions found from fragmenting a precursor ion mass selection window are compared to a database of known product ions of compounds of interest. In addition, ion traces or extracted ion chromatograms (XICs) of the product ions found from fragmenting a precursor ion mass selection window are analyzed to provide quantitative and qualitative information.

However, identifying compounds of interest in a sample analyzed using SWATH acquisition, for example, can be difficult. It can be difficult because either there is no precursor ion information provided with a precursor ion mass selection window to help determine the precursor ion that produces each product ion, or the precursor ion information provided is from a mass spectrometry (MS) observation that has a low sensitivity. In addition, because there is little or no specific precursor ion information provided with a precursor ion mass selection window, it is also difficult to determine if a product ion is convolved with or includes contributions from multiple precursor ions within the precursor ion mass selection window.

As a result, a method of scanning the precursor ion mass selection windows in SWATH acquisition, called scanning SWATH, was developed. Essentially, in scanning SWATH, a precursor ion mass selection window is scanned across a mass range so that successive windows have large areas of overlap and small areas of non-overlap. This scanning makes the resulting product ions a function of the scanned precursor ion mass selection windows. This additional information, in turn, can be used to identify the one or more precursor ions responsible for each product ion.

Scanning SWATH has been described in International Publication No. WO 2013/171459 A2 (hereinafter “the '459 Application”). In the '459 Application, a precursor ion mass selection window or precursor ion mass selection window of 25 Da is scanned with time such that the range of the precursor ion mass selection window changes with time. The timing at which product ions are detected is then correlated to the timing of the precursor ion mass selection window in which their precursor ions were transmitted.

The correlation is done by first plotting the mass-to-charge ratio (m/z) of each product ion detected as a function of the precursor ion m/z values transmitted by the quadrupole mass filter. Since the precursor ion mass selection window is scanned over time, the precursor ion m/z values transmitted by the quadrupole mass filter can also be thought of as times. The start and end times at which a particular product ion is detected are correlated to the start and end times at which its precursor is transmitted from the quadrupole. As a result, the start and end times of the product ion signals are used to determine the start and end times of their corresponding precursor ions.

### MS³ Background

Mass spectrometry/mass spectrometry/mass spectrometry (MS³) is an increasing popular technique for quantitation experiments. Like mass spectrometry/mass spectrometry (MS/MS), which is commonly used in quantitation, MS² involves selecting a precursor ion for fragmentation and monitoring the fragmentation for a first generation fragment ion, or product ion. However, MS³ includes the additional step of fragmenting the product ion and monitoring that fragmentation for one or more second generation fragment ions. This additional step gives MS³ experiments greater specificity and greater resilience to chemical noise in comparison to MS/MS experiments.

### Fragmentation Techniques Background

Electron-based dissociation (ExD), ultraviolet photodissociation (UVPD), infrared photodissociation (IRMPD) and collision-induced dissociation (CID) or CAD are often used as fragmentation techniques for tandem mass spectrometry (MS/MS). ExD can include, but is not limited to, electron-induced dissociation (EID), electron impact excitation in organics (EIEIO), electron-capture dissociation (ECD), electron activated dissociation (EAD), or electron transfer dissociation (ETD). CID is the most conventional technique for dissociation in tandem mass spectrometers.

As described above, in top-down and middle-down proteomics, an intact or digested protein is ionized and subjected to tandem mass spectrometry. ECD, for example, is a dissociation technique that dissociates peptide and protein backbones preferentially. As a result, this technique is an ideal tool to analyze peptide or protein sequences using a top-down and middle-down proteomics approach.

### SUMMARY

The teachings herein relate to identifying a compound of an experimental mass spectrum using a library or database search. More particularly the teachings herein relate to systems and methods for compressing both spectral data from the experimental mass spectrum and the library or database using a neural network encoder to improve the accuracy of the identification and reduce the compute time and memory needed to perform the identification.

The systems and methods herein can be performed in conjunction with a processor, controller, or computer system, such as the computer system of FIG. 1.

A system, method, and computer program product are disclosed for identifying a compound of an experimental spectrum. In a first step, known mass spectral data of library or database is compressed using a neural network. For example, known mass spectral data of each mass spectrum of a library of spectra corresponding to known compounds is compressed using the neural network encoder. Alternatively, known mass spectral data determined from each compound of a database of known compounds is compressed using the neural network encoder. In either case, a group of corresponding compressed known representations of mass spectral data is produced.

In a second step, mass spectral data of an experimental mass spectrum is received. In a third step, the experimental mass spectral data is compressed using the neural network encoder. A corresponding compressed experimental representation of the experimental mass spectral data is produced. In a fourth step, the experimental representation is compared to the group of known representations and each comparison is scored.

In a final step, at least one comparison with a score above a predetermined score threshold is selected. The known representation is determined from the selected at least one comparison. The known mass spectral data is determined from the known representation. Finally, the known compound is determined from the known mass spectral data. The known compound is identified as a compound of the experimental spectrum.

These and other features of the applicant's teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is an exemplary diagram showing how an encoder and decoder are used, such as an auto-encoder, that is trained against public or internal spectral libraries to generate a meaningful compressed representation of a given spectrum, in accordance with various embodiments.

FIG. 3 is an exemplary diagram showing how a neural network encoder is trained using supervised learning, in accordance with various embodiments.

FIG. 4 is a schematic diagram of a system for identifying a compound of an experimental spectrum, in accordance with various embodiments.

FIG. 5 is an exemplary flowchart showing a method for identifying a compound of an experimental mass spectrum, in accordance with various embodiments.

FIG. 6 is a schematic diagram of a system that includes one or more distinct software modules and that performs a method for identifying a compound of an experimental spectrum, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

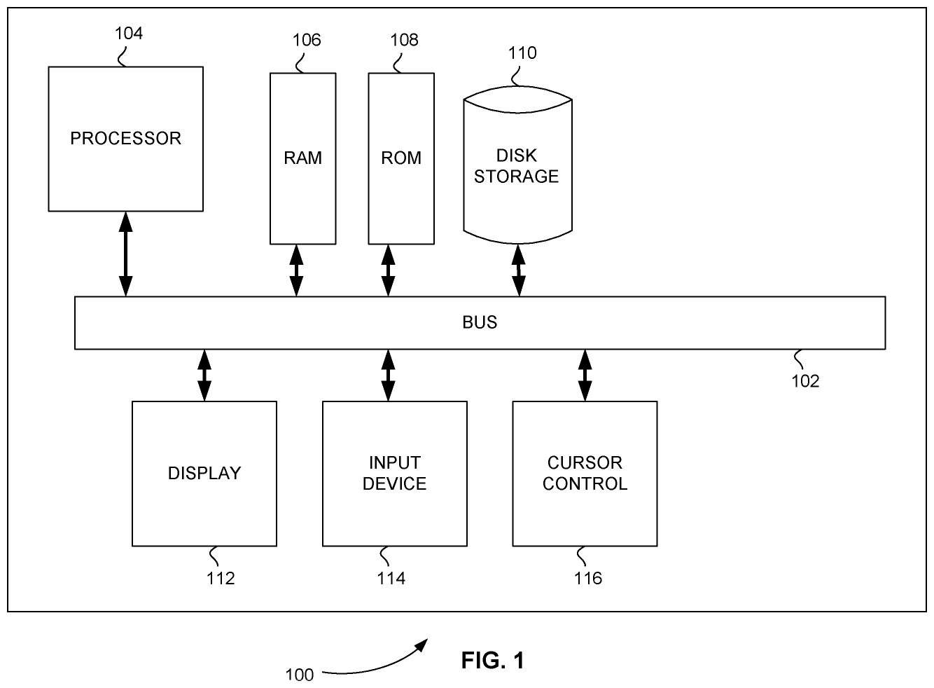

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein.

Alternatively, hard-wired circuitry may be used in place of or in combination with software instructions to implement the present teachings. For example, the present teachings may also be implemented with programmable artificial intelligence (AI) chips with only the encoder neural network programmed-to allow for performance and decreased cost. Thus, implementations of the present teachings are not limited to any specific combination of hardware circuitry and software.

The term “computer-readable medium” or “computer program product” as used herein refers to any media that participates in providing instructions to processor 104 for execution. The terms “computer-readable medium” and “computer program product” are used interchangeably throughout this written description. Such a medium may take many forms, including but not limited to, non-volatile media and volatile media. Non-volatile media includes, for example, optical or magnetic disks, such as storage device 110. Volatile media includes dynamic memory, such as memory 106.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### Identifying Compounds Using a Neural Network Encoder

As described above, tandem mass spectrometry (or mass spectrometry/mass spectrometry MS/MS) is often used to identify a compound of an experimental sample. Typically, an MS/MS or product ion spectrum is obtained. Then, some spectral data from the product ion spectrum is compared to spectral data of each mass spectrum of a library of spectra corresponding to known compounds. Alternatively, the spectral data from the product ion spectrum is compared to spectral data determined from each compound of a database of known compounds.

Each comparison of spectral data is scored. Once all the comparisons are scored, one or more compounds are selected based on the scores. The one or more selected compounds are then identified as a possible compound of the experimental sample.

MS/MS identification using library or database search works particularly well when the compounds of a sample vary significantly in chemical structure, particularly when the fragments of different compounds in the sample vary significantly. Unfortunately, when a sample becomes more complex, more and more of the compounds in the sample have similar fragments.

In recent years, there has been increasing work on MS/MS identification using library or database search for small molecules where complexity limits the quality of results achievable. In addition, the size of libraries and databases has also been increasing and current solutions require significant compute power, via the use of cloud computing, or have limited library size if run locally.

As a result, additional systems and methods are needed to improve the results and reduce the memory and computer processing needed to identify small molecules in complex experimental samples using MS/MS and library or database search.

In various embodiments, the algorithms used in library or database search are improved to minimize the compute power and thus maximize library size. Improving the spectral similarity metric or confidence, such as cosine score, to involve better chemistry or acquisition conditions also improved the results.

In various embodiments, the library search is used in real-time to enact secondary scans such as MS/MS/MS (MS3). This improves the effectiveness of the decision-making and thus increases the number of identifications and the confidence in identifications.

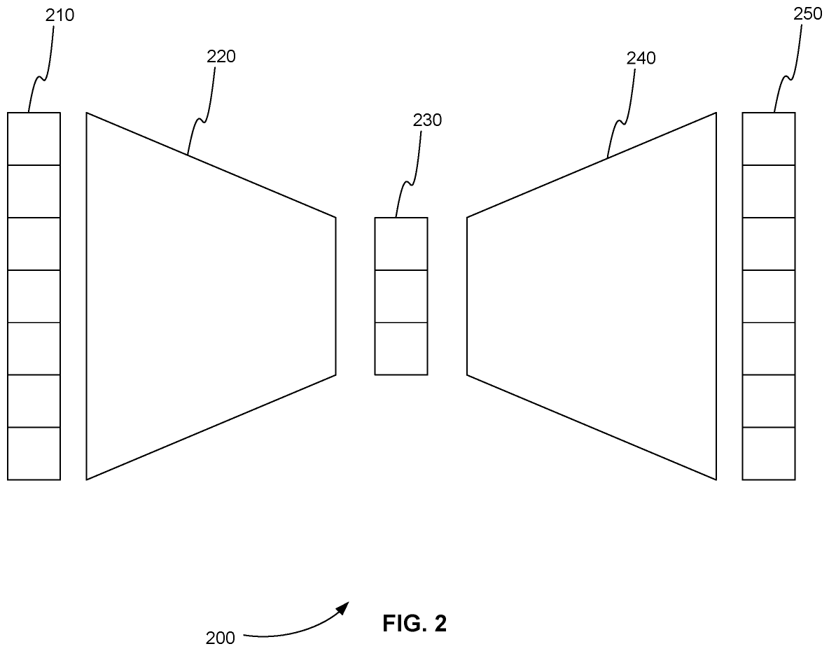

FIG. 2 is an exemplary diagram 200 showing how an encoder and decoder are used, such as an auto-encoder, that is trained against public or internal spectral libraries to generate a meaningful compressed representation of a given spectrum, in accordance with various embodiments. Input 210 to the model is the raw or binned spectra with normalized intensities or just 1 or 0 if the peak is present. For training, the output 250 is also set to be the same spectra. Neural network encoder 220 then generates compressed representation 230 of input 210. Decoder 240 can be used to decompress representation 230 of input 210. Output 250 may include other information such as chemical annotations to alter compressed representation 230 to improve scoring.

A reference library is encoded before any experimental acquisition. The encoded reference is loaded into memory in an advanced data structure such as a k-d tree, for example. This allows for O(log N) time complexity for nearest neighbor search. The nearest neighbors can then be used to calculate traditional similarity metrics such as cosine distance or spectral angle to find the best library search result. Note that the used of a k-d tree algorithm is useful until D≤20 where D in this case is the number of dimensions in compressed representation 230. O(log N) is a in best case search and O(N) is the worst case. N must be >>2^{D} to be better than O(N). Use of this algorithm is dependent on the distribution of points in library—better if randomly distributed so will need to inspect encoded space of library as well as ensure the number of dimensions D for the encoded space can successfully represent all compounds. See https://en.wikipedia.org/wiki/K-d_tree.

In various embodiments, to improve scoring at the sacrifice of performance, the compressed representation may be larger than the input data. As the input spectral vector is sparse, the main purpose of the encoder is to generate a dense vector representation which is required for advanced data structures such as k-d trees to work effectively and improve computer performance to provide real-time search. If the dense vector representation is less than the input vector, algorithm execution speed is increased further but is not required for real-time performance.

The architecture of the encoder is generic and can include any number of transformer-based layers, convolutional layers, graph neural network layers, recurrent layers, etc.

An effective bottleneck or compressed size of representation 230 can be on the order of <256 floating point numbers to represent millions of compounds effectively. The number of floating point numbers required may be as low as 16. Based on a lower boundary of 16, assuming 2 bytes to represent each number and excluding memory required to define the tree structure, it could be possible to reasonably load ˜100 million encoded spectra into memory, ˜3.2 GB, for search locally on the instrument without the need of cloud computing. Although, search time might be negatively affected at that library size so more advanced data structures might be needed to achieve real-time performance requirements on a regular CPU; this could include exact or approximate nearest neighbour search algorithms to build an index using one or more of locality-sensitive hashing (LSH), Voronoi cells, an inverted file index algorithm, or product quantization.

In various embodiments, neural network encoding is applied to achieve library or database search in the time frame of an acquisition. Conventionally, it is known that real-time database search can only be achieved with the help of a GPU cluster.

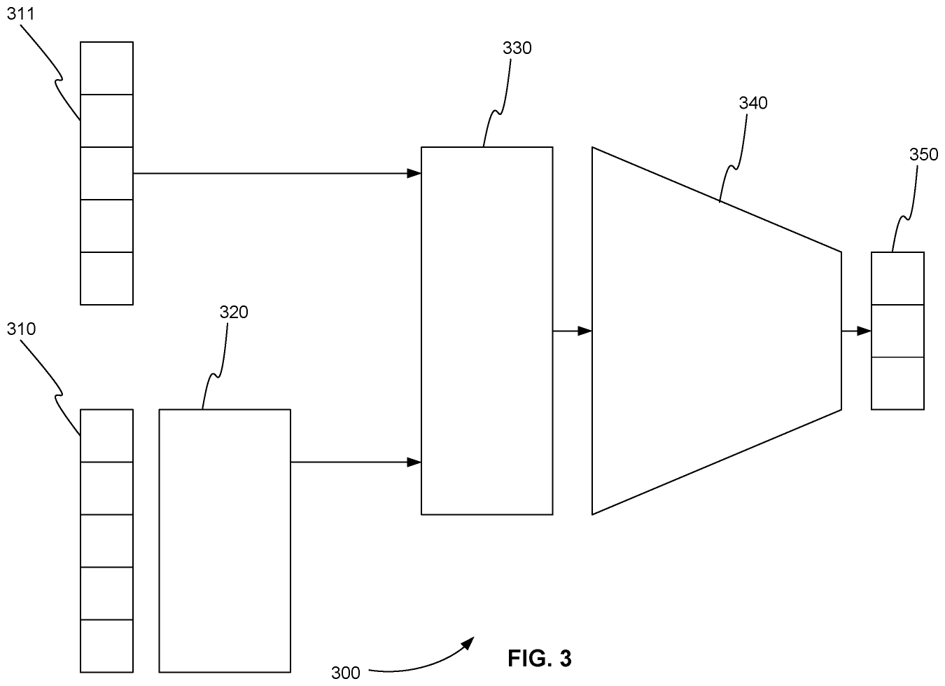

FIG. 3 is an exemplary diagram 300 showing how a neural network encoder is trained using supervised learning, in accordance with various embodiments. In FIG. 3, neural network encoder 340 is trained without the decoder in a supervised approach with contrastive learning examples. In various alternative embodiments, neural network encoder 340 is trained with a decoder as part of a deep generative model in an unsupervised approach as shown in FIG. 2.

In FIG. 3, spectral masses 310 are input into a neural network embedding layer 320 that converts the sparse one-hot encoded input to a dense vector representation. Embedding layer 320 can be trained independently in an unsupervised fashion to generate the mass embeddings.

Converted spectral masses 310 are then combined with peak confidence information 311 in combination step 330 through mathematical transformation such as standardization and scaling or vector concatenation. Peak confidence information 311 can be calculated from, for example, normalized peak intensity information, peak width, peak symmetry, or peak annotation information.

After the inputs are combined, neural network encoder 340 compresses the combined information to generate the spectral embedding at its output as in FIG. 2, for example. The use of embedding layer 320 and combination step 330 allows all required input and parameters needed to improve the scoring mechanism to be included. Meanwhile, it minimizes the size of the input using a sparse representation for 310 to reduce the training time as well as inference time; this is necessary for real-time library search as experimental spectra need to be encoded prior to comparison.

In various embodiments, the configuration shown in FIG. 3 is be trained using contrastive examples of spectral pairs with similar or dissimilar compound identities such as in a Siamese network.

In various embodiments, the number of spectral masses 310 can be limited to reduce input size further for speed: for example, spectral pre-processing may be done to remove noise peaks and select the top 1000 peaks of highest peak confidence. The input may be ordered from highest to lowest peak confidence and embedding layer 320 can consist of transformer model layers such as positional encoding and attention layers to take into account sequence information. In various embodiments, this could remove the need for combination step 330 for peak confidence.

In various embodiments, neural network encoding is used to trigger an intelligent decision-dependent event during acquisition, such as an MS3 scan. This allows for an increased overall sampling rate on the MS/MS scans and minimizes the MS3 scans required while maximizing coverage. This approach has, for example, been used in proteomics for peptide quantitation using isobaric tags to trigger on only confidently identified peptides.

In various embodiments, this workflow is used for electron-capture dissociation (ECD) applications. In these applications, library or database search is used to decide if the alternative scans need to be triggered to provide further structural elucidation rather than conducting independent runs.

In various embodiments, independent neural network encoder models are created for different instruments. Once an initial model has been generated with public or private repositories, it is fine-tuned on-the-fly or via postprocessing for subsequent runs against spectra with confident identifications to improve the latent representation. In the context of AI modelling, ‘fine-tuning’ refers to but is not limited to further training the initial model with additional data to update its parameters: additional untrained layers may be added to the model during this procedure and certain layers of the model may be fixed so a portion of the model's parameters are not affected by the additional training.

Conventionally, some papers have suggested using AI-based models in conjunction with mass spectrometry. For example, Zhou et al., Deep autoencoder for mass spectrometry feature learning and cancer detection, (2020), Faculty of Engineering and Information Sciences—Papers: Part B. 3790 (hereinafter the “Zhou Paper”) describes using machine learning models to diagnose cancer based on features learned from mass spectrometry data. The Zhou Paper, however, does not suggest using AI-based models in conjunction with library or database search.

Other papers have suggested real-time library or database search in conjunction with cloud computing, but without the use of AI-based models. For example, Barshop et al., Small molecule real-time library search, https://assets.thermofisher.com/TFS-Assets/CMD/posters/po-000115-lsms-orbitrap-iq-x-realtime-library-po000115-en.pdf, (hereinafter the “Barshop Paper”) disclose real-time library search in conjunction with mzVault spectral library access and mzCloud scoring.

Finally, some papers have described alternative methods for scoring library or database search. However, these methods do not use autoencoders or transformer-based layers. They also do not try to integrate an embedding layer and peak confidence to achieve increased training and inference performance required for real-time processing.

For example, Huber et al, Spec2vec: improved mass spectral similarity scoring through the learning of structural relationships, published in PLOS Computational Biology, February 2021 (hereinafter the “Huber Paper1”) simply discloses a spectral similarity scoring method inspired by natural language processing that trains a shallow 2-layer neural network model to generate an embedding to better represent the masses. These are then weighted-averaged using the intensities to create a more favorable spectral representation for scoring. Its AI model is not directly used to generate the spectral representation and does not, for example, use intensities as an input. Thus, it cannot learn from the spectra's intensity pattern.

Huber et al, MS2DeepScore: A novel Deep Learning Similarity Measure To Compare Tandem Mass Spectra, J Cheminform 13, 84 (2021) (hereinafter the “Huber Paper2”) discloses a 2 feed-forward dense layer Siamese network which does take the spectra with its intensities as an input but uses contrastive training to learn a spectral embedding for scoring using the chemical structural similarity as a target in its loss function. Unlike autoencoders and other deep generative models, this is a supervised approach and requires labelled data. Similar to Huber Paper1, the Huber Paper2 is directed to improving spectral similarity measures and not directed to improve computing performance. As it does not include an embedding layer and requires labelled data, its application scope is limited with spectra binned to 0.01 Da resolution for example and a shallow 2 layer neural network.

The described above, various embodiments in FIG. 2 and FIG. 3 address these multiple limitations in the AI model to maximize performance while retaining and improving the scoring mechanism. In addition, neither Huber1 and Huber2 use an AI model in conjunction with an advanced data structure such as k-d tree to achieve real-time search for large library sizes.

### System for Identifying a Compound

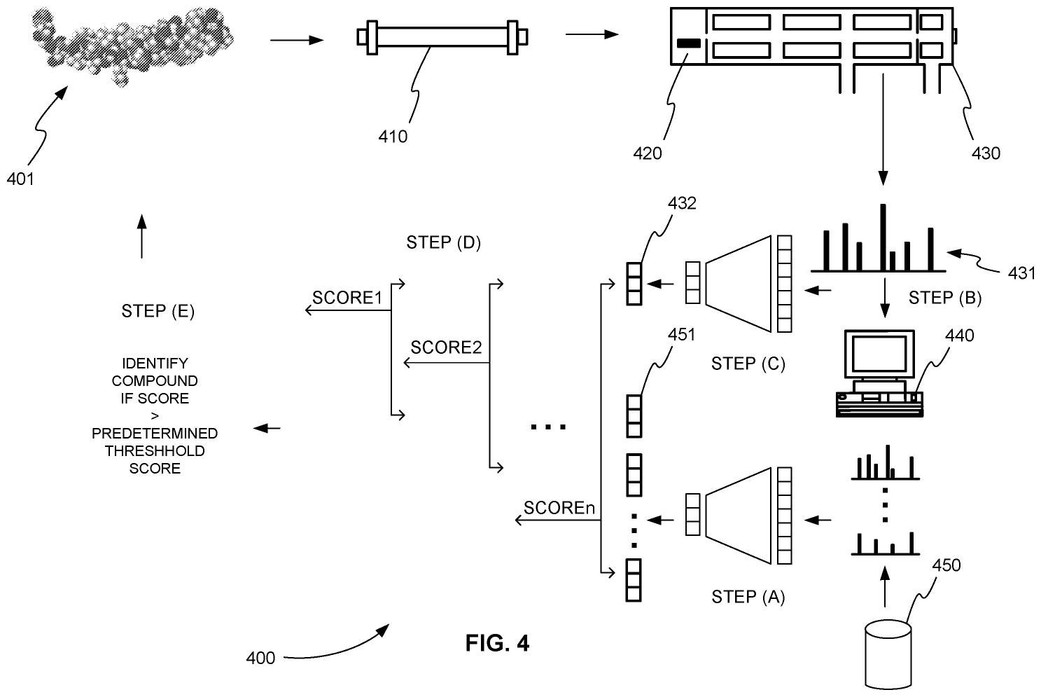

FIG. 4 is a schematic diagram 400 of a system for identifying a compound of an experimental spectrum, in accordance with various embodiments. The system includes processor 440. Processor 440 can be, but is not limited to, a controller, a computer, a microprocessor, the computer system of FIG. 1, or any device capable of analyzing data. Processor 440 can also be any device capable of sending and receiving control signals and data.

In a step (A), processor 440 compresses known mass spectral data of library or database 450 using a neural network encoder. For example, (i) processor 440 compresses known mass spectral data of each mass spectrum of a library of spectra corresponding to known compounds using the neural network encoder. Alternatively, (ii) processor 440 compresses known mass spectral data determined from each compound of a database of known compounds using the neural network encoder. In either case, a group 451 of corresponding compressed known representations of mass spectral data is produced.

In a step (B), processor 440 receives mass spectral data of an experimental mass spectrum 431. In a step (C), processor 440 compresses the experimental mass spectral data using the neural network encoder. A corresponding compressed experimental representation 432 of the experimental mass spectral data is produced.

In a step (D), processor 440 compares experimental representation 432 to group 451 of known representations and scores each comparison.

In a step (E), processor 440 selects at least one comparison with a score above a predetermined score threshold. The known representation is determined from the selected at least one comparison. The known mass spectral data is determined from the known representation. Finally, the known compound is determined from the known mass spectral data. The known compound is identified as a compound 401 of experimental spectrum 431.

In various embodiments, the library includes unknown spectra.

In various embodiments, mass spectral data is product ion data. Specifically, known mass spectral data of each mass spectrum of the library comprises product ion mass spectral data. Known mass spectral data determined from each compound of the database comprises product ion mass spectral data. Also, experimental mass spectral data of experimental mass spectrum 431 comprises product ion mass spectral data.

In various embodiments, representations are stored in less memory than their corresponding mass spectral data. Specifically, each known representation of the group of known representations is stored in less memory than corresponding known mass spectral data of each mass spectrum of the library or corresponding known mass spectral data determined from each compound of the database. Also, experimental representation 432 is stored in less memory than the experimental mass spectral data.

In various embodiments, the mass spectral data is mass-to-charge ratio (m/z) data. Specifically, known mass spectral data of each mass spectrum of the library includes m/z data. Known mass spectral data determined from each compound of the database includes m/z data. Also, experimental mass spectral data of experimental mass spectrum 431 comprises m/z data.

In various embodiments, the mass spectral data can further include, but is not limited to including, intensity, retention time, and precursor ion data. In addition, it can also include any information related to peak confidence e.g., peak width, peak symmetry etc. It may also include peak chemical annotations such as if it is mono-isotope, isotope number, charge state or whether it is a loss. Specifically, known mass spectral data of each mass spectrum of the library further includes one or more of intensity, retention time, and precursor ion data. Known mass spectral data determined from each compound of the database includes one or more of intensity, retention time, and precursor ion data. Also, experimental mass spectral data of experimental mass spectrum 431 includes one or more of intensity, retention time, and precursor ion data.

In various embodiments, the mass spectral data can include one or multiple spectra from different instrument acquisition settings. For example, it could include a CID MS/MS spectrum and an ExD MS/MS spectra acquired for the same compound. This would be compressed to a fixed length vector for comparison with a library of previously acquired CID and ExD spectra pairs.

In various embodiments, each known representation of group 451 of known representations includes a compressed vector of numbers, and experimental representation 432 includes a compressed vector of numbers.

In various embodiments, experimental representation 432 is compared to group 451 of representations using one or more of a tree data structure, locality-sensitive hashing (LSH), Voronoi cells, an inverted file index algorithm, or product quantization.

In various embodiments, steps (B)-(E) are performed post-acquisition. Alternatively, in various embodiments, steps (B)-(E) are performed in real-time and within an acquisition time period of a sample.

In various embodiments, at least one secondary scan of the sample is triggered during or after step (D) or after step (E).

In various embodiments, the at least one secondary scan of the sample is triggered during or after step (D) and results from the at least one secondary scan are used in scoring at least one comparison of step (D).

In various embodiments, wherein multiple scans are triggered to improve identification confidence

[symbol]

In various embodiments, the at least one secondary scan comprises a mass spectrometry/mass spectrometry/mass spectrometry (MS3) scan, an electron-capture dissociation (ECD) scan, or a scan with a different collision energy, switching polarity, charge state, or precursor ion window.

In various embodiments, the experimental mass spectra being compared or the library mass spectrum consists of one or more spectra acquired with different acquisition settings (e.g., ECD, MS3, EAD, etc.).

In various embodiments, at least one secondary scan is triggered in a situation where the identification of the compound through the scoring mechanism is ambiguous. For, example, when the identified compound has low confidence or if multiple compounds have similar high confidence, additional supporting information through the secondary scan is required to confirm or reject identification.

In various embodiments, the neural network encoder or certain subset of the model is trained using one or more public repositories such as the global natural product social molecular networking (GNPS) or the national institute of standards and technology (NIST) mass spectral library to generate an initial model. It is then fine-tuned on smaller customer-specific spectral libraries to improve scoring performance.

In various embodiments, the neural network encoder is trained with a decoder as an auto-encoder.

In various embodiments, the encoder or decoder output or loss function comprises the model's input spectral data.

In various embodiments, the encoder or decoder output or loss function comprises chemical identity information.

In various embodiments, the neural network encoder is trained using a contrastive training method in a supervised learning fashion.

In various embodiments, the neural network encoder initially trained using a deep generative unsupervised method is fine-tuned using a supervised contrastive training method.

In various embodiments, a portion or the complete deep generative model is used with additional neural network layers added while fine-tuning for the supervised training method.

In various embodiments, the initial unsupervised model is fixed and only additional neural network layers are fine-tuned while training.

In various embodiments, the neural network encoder comprises neural network layers that account for the position of data points in sequential input data such as transformers and recurrent neural networks.

In various embodiments, the neural network encoder or a portion of the model is trained using an initial spectral library and then fine-tuned on a secondary library.

In various embodiments, the initial library can be a combination of one or more of the global natural product social molecular networking (GNPS) spectral library and the national institute of standards and technology (NIST) mass spectral library.

In various embodiments, the initial library comprises in-silico generated or simulated spectral data.

In various embodiments, the neural network encoder has an input embedding layer to encode the spectral peak data prior to additional neural network layers that compress to create the spectral encoding.

In various embodiments, after the mass embedding layer, peak mass metadata is combined through a mathematical transformation, scaling or concatenation to the embedded vector of numbers.

In various embodiments, the peak metadata comprises one or more of peak intensity, peak width, and peak symmetry.

In various embodiments, the peak metadata includes a chemical annotation including if it is isotope or a loss.

In various embodiments, the spectral metadata such as precursor ion data or retention time is concatenated to the output of encoder prior to searching the library of encoded spectra.

In various embodiments, the at least one secondary scan comprises a mass spectrometry/mass spectrometry/mass spectrometry (MS3) scan, an electron-capture dissociation (ECD) scan, or a scan with a different collision energy, switching polarity, charge state, or precursor ion window.

In various embodiments, the neural network encoder or certain subset of the model is trained initially using a deep generative approach in an unsupervised fashion on large unlabelled data. It is then fine-tuned on smaller libraries of labelled examples in a supervised method such as with contrastive training and examples as not as much of this data would be available.

In various embodiments, the system of FIG. 4 further includes mass spectrometer 430 that measures mass spectrum 431 and sends mass spectrum 431 to processor 440. Ion source device 420 of mass spectrometer 430 ionizes separated fragments of compound 401 or only compound 401, producing an ion beam. Ion source device 420 is controlled by processor 440, for example. Ion source device 420 is shown as a component of mass spectrometer 430. In various alternative embodiments, ion source device 420 is a separate device. Ion source device 420 can be, but is not limited to, an electrospray ion source (ESI) device or a chemical ionization (CI) source device such as an atmospheric pressure chemical ionization source (APCI) device or an atmospheric pressure photoionization (APPI) source device.

Mass spectrometer 430 mass analyzes product ions of compound 401 or selects and fragments compound 401 and mass analyzes product ions of compound 401 from the ion beam at a plurality of different times. Mass spectrum 431 is produced for compound 401. Mass spectrometer 430 is controlled by processor 440, for example.

In the system of FIG. 4, mass spectrometer 430 is shown as a triple quadrupole device. One of ordinary skill in the art can appreciate that any component of mass spectrometer 430 can include other types of mass spectrometry devices including, but not limited to, ion traps, orbitraps, time-of-flight (TOF) devices, ion mobility devices, or Fourier transform ion cyclotron resonance (FT-ICR) devices.

In various embodiments, the system of FIG. 4 further includes additional device 410 that affects compound 401 providing the at least one additional dimension. As shown in FIG. 4, additional device 410 is an LC device and the at least one additional dimension or spectral data provided is retention time. In various alternative embodiments. additional device 410 can be, but is not limited to, a gas chromatography (GC) device, capillary electrophoresis (CE) device, an ion mobility spectrometry (IMS) device, or a differential mobility spectrometry (DMS) device. In still further embodiments, additional device 410 is not used and the at least one additional dimension or spectral data provided is precursor ion m/z and is provided by mass spectrometer 430 operating in a precursor ion scanning mode.

### Method for Identifying a Compound

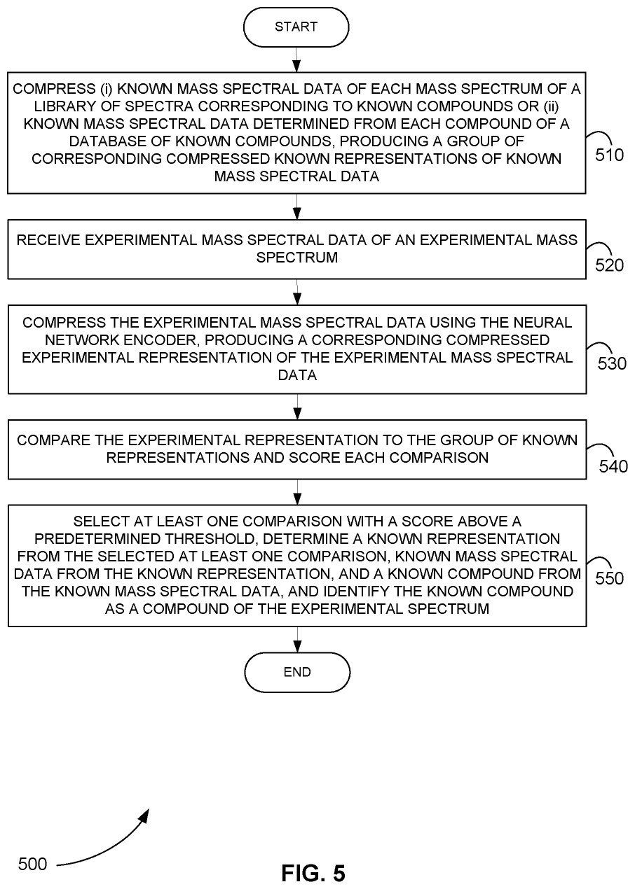

FIG. 5 is an exemplary flowchart showing a method 500 for identifying a compound of an experimental mass spectrum, in accordance with various embodiments.

In step 510 of method 500, (i) known mass spectral data of each mass spectrum of a library of spectra corresponding to known compounds or known mass spectral data determined from each compound of a database of known compounds are compressed using a neural network encoder. A group of corresponding compressed known representations of known mass spectral data is produced.

In step 520, experimental mass spectral data of an experimental mass spectrum is received.

In step 530, the experimental mass spectral data is compressed using the neural network encoder. A corresponding compressed experimental representation of the experimental mass spectral data is produced.

In step 540, the experimental representation is compared to the group of known representations and each comparison is scored.

In step 550, at least one comparison with a score above a predetermined score threshold is selected. A known representation is determined from the selected at least one comparison. A known mass spectral data is determined from the known representation. A known compound is determined from the known mass spectral data. Finally, the known compound is identified as a compound of the experimental spectrum.

### Computer Program Product for Identifying a Compound

In various embodiments, a computer program product includes a non-transitory tangible computer-readable storage medium whose contents include a program with instructions being executed on a processor so as to perform a method for identifying a compound of an experimental spectrum. This method is performed by a system that includes one or more distinct software modules.

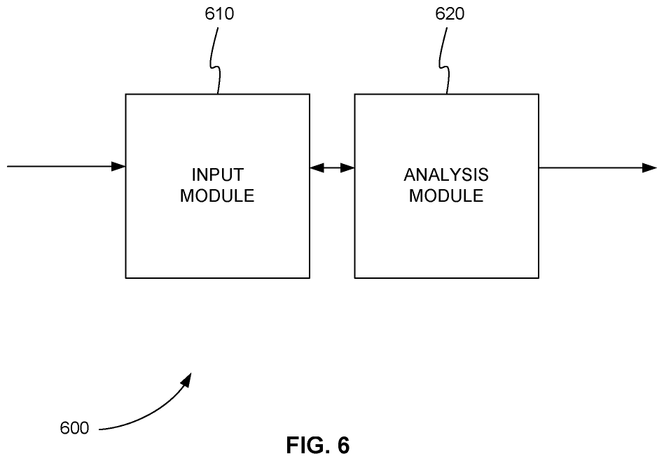

FIG. 6 is a schematic diagram of a system 600 that includes one or more distinct software modules and that performs a method for identifying a compound of an experimental spectrum, in accordance with various embodiments. System 600 includes input module 610 and analysis module 620.

Analysis module 620 compresses (i) known mass spectral data of each mass spectrum of a library of spectra corresponding to known compounds or (ii) known mass spectral data determined from each compound of a database of known compounds using a neural network encoder. A group of corresponding compressed known representations of known mass spectral data is produced.

Input module 610 receives experimental mass spectral data of an experimental mass spectrum.

Analysis module 620 compresses the experimental mass spectral data using the neural network encoder. A corresponding compressed experimental representation of the experimental mass spectral data is produced. Analysis module 620 compares the experimental representation to the group of known representations and scores each comparison.

Analysis module 620 selects at least one comparison with a score above a predetermined score threshold. Analysis module 620 determines a known representation from the selected at least one comparison, known mass spectral data from the known representation, and a known compound from the known mass spectral data. Analysis module 620 identifies the known compound as a compound of the experimental spectrum.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A method for identifying a compound of an experimental mass spectrum, comprising:
(a) compressing
(i) known mass spectral data of each mass spectrum of a library of spectra corresponding to known compounds or
(ii) known mass spectral data determined from each compound of a database of known compounds
using a neural network encoder, producing a group of corresponding compressed known representations of known mass spectral data;
(b) receiving experimental mass spectral data of an experimental mass spectrum;
(c) compressing the experimental mass spectral data using the neural network encoder, producing a corresponding compressed experimental representation of the experimental mass spectral data;
(d) comparing the experimental representation to the group of known representations and scoring each comparison; and
(e) selecting at least one comparison with a score above a predetermined score threshold, determining a known representation from the selected at least one comparison, known mass spectral data from the known representation, and a known compound from the known mass spectral data, and identifying the known compound as a compound of the experimental spectrum.

2. The method of claim 1, wherein known mass spectral data of each mass spectrum of the library comprises product ion mass spectral data, known mass spectral data determined from each compound of the database comprises product ion mass spectral data, and experimental mass spectral data of the experimental mass spectrum comprises product ion mass spectral data or wherein the library comprises unknown spectra.

3. The method of claim 1, wherein each known representation of the group of known representations is stored in less memory than corresponding known mass spectral data of each mass spectrum of the library or corresponding known mass spectral data determined from each compound of the database and wherein the experimental representation is stored in less memory than the experimental mass spectral data.

4. The method of claim 1, wherein known mass spectral data of each mass spectrum of the library further comprises one or more of intensity, retention time, and precursor ion data, known mass spectral data determined from each compound of the database comprises one or more of intensity, retention time, and precursor ion data, and experimental mass spectral data of the experimental mass spectrum comprises one or more of intensity, retention time, and precursor ion data.

5. The method of claim 1, wherein the experimental representation is compared to the group of representations using one or more of a tree data structure, locality-sensitive hashing (LSH), Voronoi cells, an inverted file index algorithm, or product quantization.

6. The method of claim 1, wherein the neural network encoder is initially trained using a deep generative unsupervised method and is fine-tuned using a supervised training method with or without additional neural network layers, or wherein the neural network encoder is trained with a decoder as an auto-encoder, or wherein the neural network encoder comprises neural network layers that account for the position of data points in sequential input data such as transformers and recurrent neural networks.

7. The method of claim 1, wherein the neural network encoder has an input embedding layer to encode spectral peak data prior to additional neural network layers that compress to create the spectral encoding or wherein an encoder or decoder output or loss function comprises input spectral data and chemical identity information of a model of the neural network encoder.

8. The method of claim 1, wherein the library comprises in-silico generated or simulated spectral data or wherein the neural network encoder or a portion of a model of the neural network encoder is trained using an initial spectral library and then finetuned on a secondary library.

9. The method of claim 7, wherein after the mass input embedding layer, peak mass metadata is combined through a mathematical transformation, scaling, or concatenation to an embedded vector of numbers.

10. The method of claim 9, wherein the peak mass metadata comprises peak confidence data or a chemical annotation or wherein spectral metadata such as precursor ion data or retention time is concatenated to the output of the encoder prior to searching the library of encoded spectra.

11. The method of claim 1, wherein at least one secondary scan comprises a mass spectrometry/mass spectrometry/mass spectrometry (MS3) scan, an electron-based dissociation (ExD) scan, or a scan with a different collision energy, switching polarity, charge state, or precursor ion window or wherein the experimental mass spectra being compared or the library mass spectrum consists of one or more spectra acquired with different acquisition settings.

12. The method of claim 11, wherein steps (b)-(e) are performed post-acquisition or in real-time and within an acquisition time period of a sample.

13. The method of claim 1, wherein at least one secondary scan of the sample is triggered during or after step (d) or after step (e) and results from the at least one secondary scan are used in scoring at least one comparison of step (d) or wherein multiple scans are triggered to improve identification confidence.

14. A computer program product, comprising a non-transitory tangible computer-readable storage medium whose contents cause a processor to perform a method for identifying a compound of an experimental spectrum, comprising:
providing a system, wherein the system comprises one or more distinct software modules, and wherein the distinct software modules comprise an input module and an analysis module;
compressing
(i) known mass spectral data of each mass spectrum of a library of spectra corresponding to known compounds or
(ii) known mass spectral data determined from each compound of a database of known compounds
using a neural network encoder using the analysis module, producing a group of corresponding compressed known representations of known mass spectral data;
receiving experimental mass spectral data of an experimental mass spectrum using the input module;
compressing the experimental mass spectral data using the neural network encoder using the analysis module, producing a corresponding compressed experimental representation of the experimental mass spectral data;
comparing the experimental representation to the group of known representations and scoring each comparison using the analysis module; and
selecting at least one comparison with a score above a predetermined score threshold, determining a known representation from the selected at least one comparison, known mass spectral data from the known representation, and a known compound from the known mass spectral data, and identifying the known compound as a compound of the experimental spectrum using the analysis module.

15. A system for identifying a compound of an experimental spectrum, comprising:
a processor that
compresses
(i) known mass spectral data of each mass spectrum of a library of spectra corresponding to known compounds or
(ii) (ii) known mass spectral data determined from each compound of a database of known compounds
using a neural network encoder, producing a group of corresponding compressed known representations of mass spectral data,
receives mass spectral data of an experimental mass spectrum;
compresses the experimental mass spectral data using the neural network encoder, producing a corresponding compressed experimental representation of the
experimental mass spectral data, compares the experimental representation to the group of known representations and scores each comparison, and
selects at least one comparison with a score above a predetermined score threshold, determines a known representation from the selected at least one comparison, known mass spectral data from the known representation, and a known compound from the known mass spectral data, and identifies the known compound as a compound of the experimental spectrum.
